(19) United States
(12) Patent Application Publication
(10) Pub. No.: US 2006/0211996 A1
(43) Pub. Date: Sep. 21, 2006

(54) Controlled flow adapter for medical fluid containers

(75) Inventors: James A. Trinchera, Encinitas, CA (US); Philip J. Simpson, Escondido, CA (US); David G. Matsuura, Encinitas, CA (US); Taras Worona, Etobicoke (CA); Mark Costa, Milton (CA); Davis A.R. Kanbergs, Milton (CA)

Correspondence Address: BUCHANAN, INGERSOLL & ROONEY PC, POST OFFICE BOX 1404, ALEXANDRIA, VA, 22313-1404, US

(73) Assignee: VASOGEN IRELAND LIMITED, County Clare (IE)

(21) Appl. No.: 11/375,446

(22) Filed: Mar. 15, 2006

Related U.S. Application Data
(60) Provisional application No. 60/662,324, filed on Mar. 15, 2005.

(51) Int. Cl. A61M 5/00

(52) U.S. Cl. 604/246

(57) ABSTRACT

An apparatus for controlled flow of medical fluid materials for use as an adapter in place of a septum in an outlet of a medical fluid container, has a body with a hollow interior, a first open end adapted for fluid tight fit with the outlet of the medical fluid container, and a second open end adapted for fit with a medical accessory. A fluid flow control means is disposed with the body for controlling the flow of medical fluid materials through the hollow interior between the medical fluid container and the medical accessory. The fluid flow control means comprises a valve member, a valve seat, and biasing means operating on the valve member to bias the valve member against the valve seat for fluid tight fit. The valve member is motivated to open fluid communication between the medical fluid container and the medical accessory when the medical accessory is attached to the second open end and the valve member is motivated to close fluid communication between the medical fluid container and the medical accessory when the medical accessory is disengaged from the second open end.

### CROSS-REFERENCE TO RELATED APPLICATIONS

This application claims priority to U.S. Provisional Application Ser. No. 60/662324, filed Mar. 15, 2005.

### BACKGROUND OF THE INVENTION

1. Field of the Invention

The present invention relates to an apparatus for use with medical fluid containers for transfer of medical fluid materials, and more particularly a controlled flow adapter.

2. Background Art

Many different types of fluids are used by doctors and medical professionals in treatment of patients, surgeries, and other therapies. For example, drugs can be stored in bottles or vials, and fluids such as physiological saline are typically stored into bags or bottles. Accordingly, these fluids are placed in containers that maintain sterility but also permit access to the fluids by the medical professional or user. Typically, the outlet end of these containers (i.e. bottle, vial, or bag) comprise an elastomeric septum that is pierced by a needle or similar device attached to a medical accessory for insertion/withdrawal of the medical fluid to/from the container.

However, there are some drawbacks relating to the septums currently used for most medical fluid containers. Firstly, the use of needles or sharps to puncture the septum for withdrawal of the fluid is potentially dangerous to the medical professional due to the risk of needle sticks. This can cause potentially serious injury, especially if the medical fluid is toxic or poses a biohazard risk. There is a trend within medical facilities to go away from the use of needles or sharps, so as to reduce the risk of injury to both staff and patients. Secondly, the integrity of the septum can be compromised, thereby compromising sterility, by repeated insertion and withdrawal of needles. Thirdly, there is the risk that parts of the septum can break off and fall into the medical fluid when the needle is inserted In addition, there is the risk that the septum can lose its integrity (i.e. septum becomes more brittle) thus compromising the sterility of the medical fluid, if the medical fluid containers are stored for a long time.

In view of the foregoing, there is a need to have an apparatus for use with medical fluid containers for transfer of medical fluid materials, and more particularly a controlled flow adapter for use instead, or in replacement, of septums that reduces the risk of needle sticks to the medical profession, as well as maintaining the sterility of the medical fluids or preventing exposure to the environment, medical personnel, or patients to the medical fluid materials. Accordingly, it is an object of the present invention to provide a novel controlled flow adapter for use with medical fluid containers.

### SUMMARY OF THE INVENTION

According to one embodiment of this invention, a controlled flow adapter for use in place of a septum in the outlet of a medical fluid container is provided, comprising a body having a hollow interior and first and second open ends. The first open end is adapted for fluid tight fit with the outlet of the medical fluid container. The second open end is adapted for fit with a medical accessory for flow of medical fluid materials from the medical fluid container to the medical accessory. A fluid flow control means is disposed within the body for controlling flow of medical fluid materials between the hollow interior of the medical fluid container and the medical accessory. The fluid flow control means comprises a valve member, a valve seat, and biasing means operating on the valve member to bias the valve member against the valve seat for fluid tight fit. The valve member is motivated to open fluid communication between the medical fluid container and the medical accessory by attachment of the medical accessory to the second end of the body and the valve member is motivated to close fluid communication between the medical fluid container and the medical accessory when the medical accessory is disengaged from the second end of the body.

Suitably, the biasing means comprises a spring. The second end for fitting with the medical accessory preferably includes an upstanding circumferential wall, and an attachment means for releasably attaching the medical accessory to the second end. Suitably, the attachment means comprises at least one bayonet slot.

The valve member may include a valve nose extending axially outwardly from the valve seat for engagement with the medical accessory to open fluid communication. Suitably, the valve seat includes an upstanding annular ring on the surface distal to the hollow interior for fluid tight sealing engagement with the medical accessory.

According to another embodiment of the invention, there is a medical fluid container assembly having a housing for containing medical fluid, where the housing has a hollow interior and an outlet for exit of medical fluid from the hollow interior. A fluid flow control means is disposed within the outlet with fluid tight fit and is adapted for fit with a medical accessory for controlling the flow of medical fluid between the hollow interior and the medical accessory. The fluid flow control means comprises a valve member, a valve seat, and biasing means operating on the valve member to bias the valve member against the valve seat for fluid tight fit, thus providing a self-sealing function. The valve member is motivated to open fluid communication between the medical fluid container and the medical accessory by attachment of the medical accessory to the fluid flow control means and the valve member is motivated to close fluid communication between the medical fluid container and the medical accessory when the medical accessory is disengaged from the fluid flow control means.

A specific preferred embodiment of the invention is illustrated in the accompanying drawings.

### BRIEF DESCRIPTION OF THE DRAWINGS

FIG. 1 is a perspective view of the preferred embodiment of the invention;

FIG. 2 is a side view of the apparatus of FIG. 1;

FIG. 3 is a side view of the apparatus of FIG. 1 engaged at one end with a medical accessory and at the other end is mounted within the outlet of a medical fluid container; and

FIG. 4 is a cross-sectional view along the line 4-4 of FIG. 3

### DESCRIPTION OF THE PREFERRED EMBODIMENT

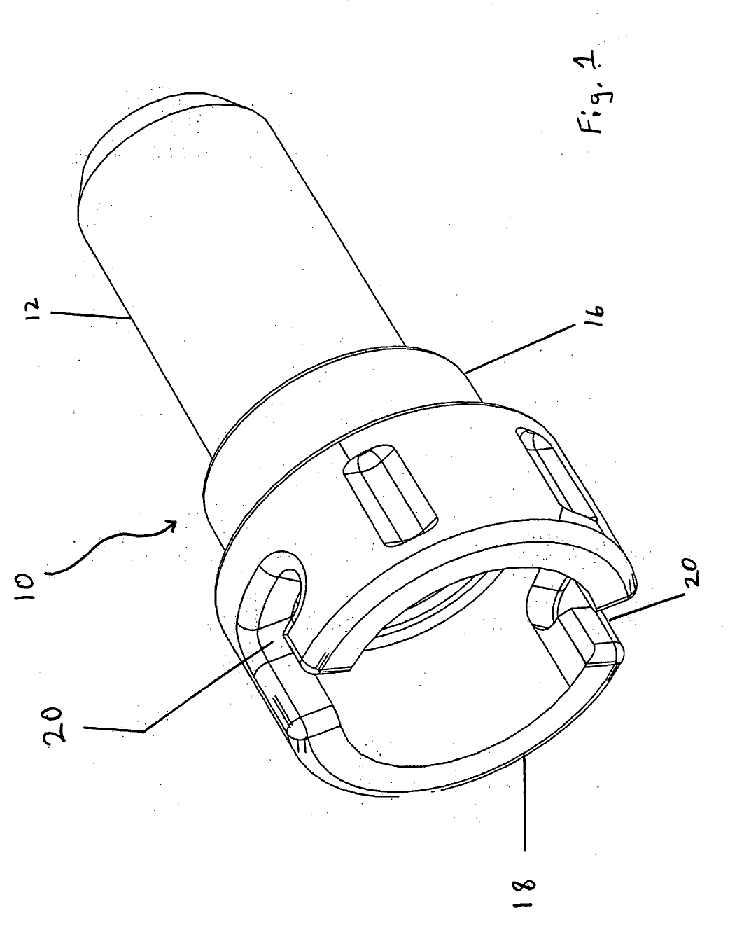

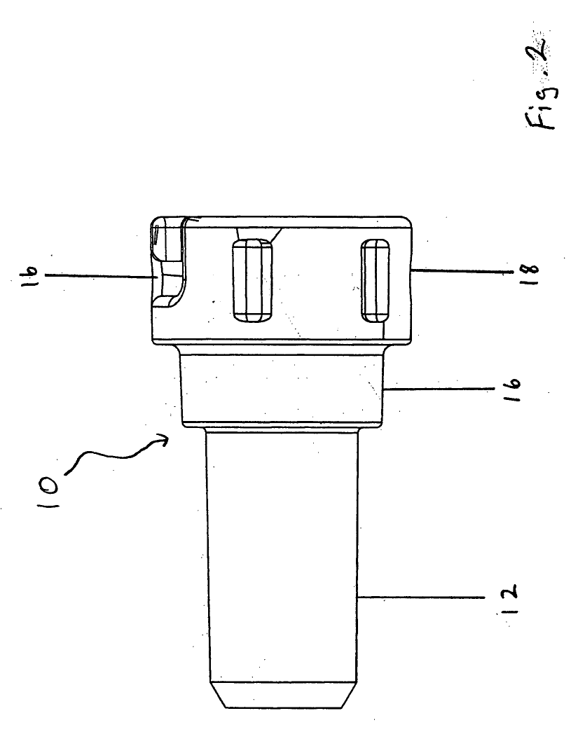

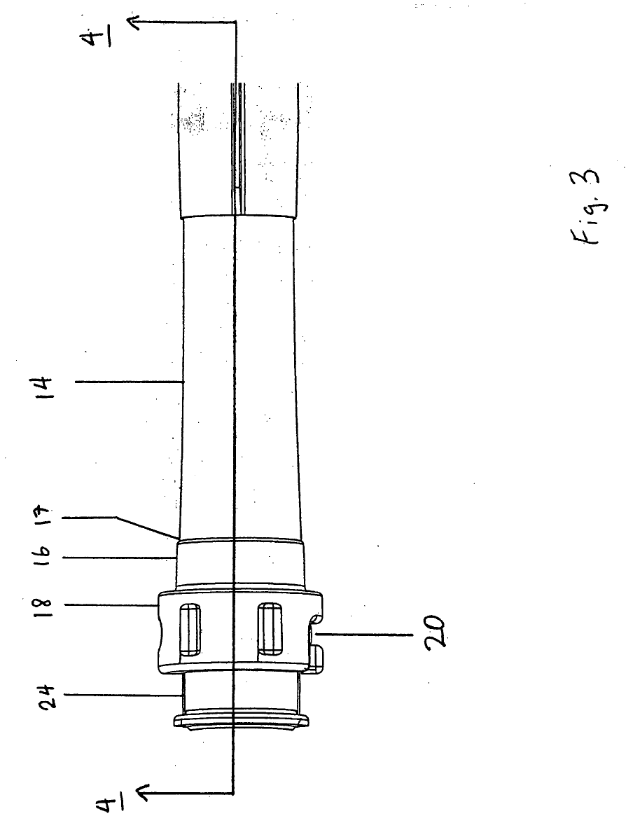

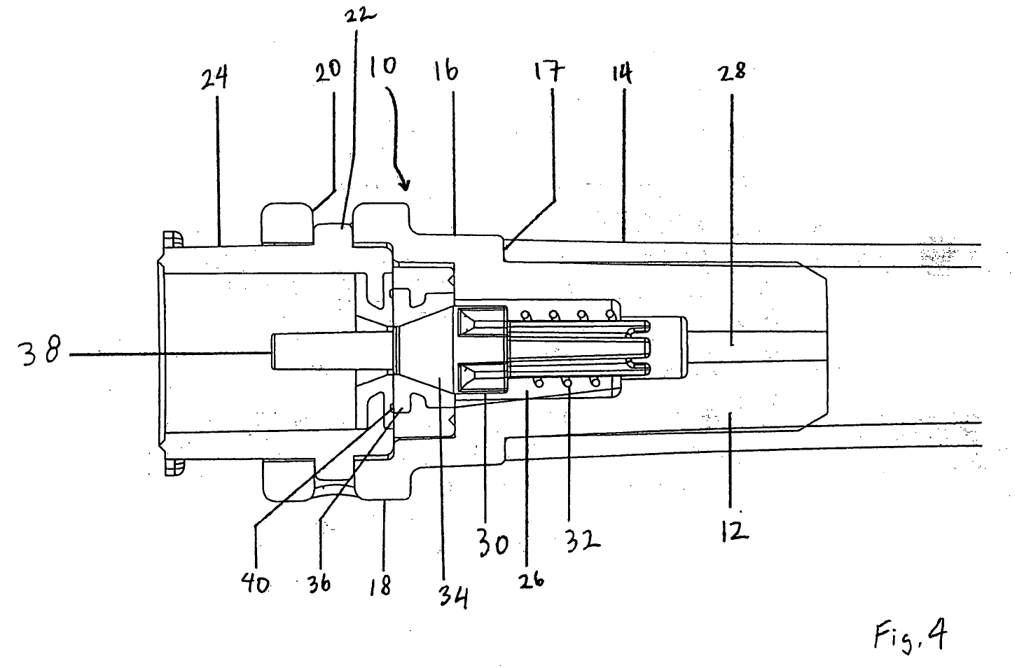

FIGS. 1 and 2 show a controlled flow adapter according to the preferred embodiment of the invention. The controlled flow apparatus 10 has an inlet housing 12 adapted for fluid tight fit with the outlet 14 (see FIG. 4) of a medical fluid container. It is to be appreciated that any suitable means of making a fluid tight connection between inlet housing 12 and outlet 14 can be used, such as adhesives, heat sealing, and other methods known in the art. A circumferential wall 16 depends from inlet housing 12 and has an outer diameter greater than the outer diameter of inlet housing 12. Circumferential wall 16 abuts against the end 17 of outlet 14 distal to the container portion of the medical fluid container (see FIGS. 3 and 4) when controlled flow adapter 10 is inserted into outlet 14. An outlet housing 18 depends from circumferential wall 16 and has an outer diameter greater than the outer diameter of circumferential wall 16. Outlet housing 18 has bayonet slots 20 for fit with corresponding bayonet pins 22 on medical accessory 24 (see FIG. 4) to releasably attach a medical accessory 24 to the controlled flow adapter 10.

As seen in FIG. 4, the controlled flow adapter 10 has a hollow interior 26 through which medical fluid materials will flow from a medical fluid container (not shown) via outlet 14 by way of an axial bore 28 within inlet housing 12. A valve 30 including a valve head 34 and a valve seat 36 is disposed within hollow interior 26, in association with a spring 32. Spring 32 biases valve head 34 against valve seat 36. Valve head 34 also has an axially extending valve nose 38 which, in operation, will engage with a valve actuating member (not shown) associated with medical accessory 24 with subsequent movement of valve 30 in a rearward direction towards the medical fluid container, thereby unseating valve head 34 from valve seat 36 to provide for fluid communication from the medical container to medical accessory 24. When medical accessory 24 is disengaged from outlet housing 18, spring 32 biases valve head 34 against valve seat 36 to provide for a fluid tight seal to prevent medical fluid materials from exiting controlled flow adapter 10 when medical accessory 24 is disengaged.

Valve seat 36 is preferably made of elastomeric material but can be made of any suitable material known in the art. An upstanding annular ring 40 is disposed on the surface of valve seat 36 distal to outlet 14 and assists in creating a fluid tight seal between valve seat 36 and medical accessory 24 when medical accessory 24 is attached to outlet housing 18, thereby reducing the risk that medical fluid will leak from the controlled flow adapter 10—medical accessory 24 interface area.

As can be appreciated, there can be modifications to the structures and functions described above. Although bayonet slots 22 are described for releasably attaching medical accessory 24 with outlet housing 18, any suitable means for releasably attaching medical accessory 24 with outlet housing 18 can be used. Although inlet housing 12 and outlet housing 18 are shown as being integrally formed, they can be made as parts that are attached together. Further, medical fluid containers can encompass any container suitable for holding or storing medical fluids such as bottle, vials, or bags made of elastomeric materials, and can encompass containers which contain lyophilized or powdered drugs which can be reconstituted with the addition of water or any suitable liquid. Medical fluid materials can encompass any fluids used for medical purposes or in a medical setting, such as physiological saline, liquid pharmaceutical products, and liquid nutritional supplements, and includes lyophilized or powdered materials that are reconstituted by the addition of water or any suitable liquid. Medical accessories can encompass any suitable device for the transfer and/or storage of medical fluid materials to or from a medical fluid container such as syringes, pumps, bottles, vials or the like.

1. An apparatus for controlled flow of medical fluid materials for use as an adapter in an outlet of a medical fluid container, comprising:
a body having a hollow interior, a first open end adapted for fluid tight fit with an outlet of said medical fluid container, and a second open end adapted for fit with a medical accessory; and
a fluid flow control means disposed within said body for controlling the flow of medical fluid materials through said hollow interior between said medical fluid container and said medical accessory;
said fluid flow control means comprising a valve member, a valve seat, biasing means operating on the valve member to bias the valve member against the valve seat for fluid tight fit, and whereby the valve member is motivated to open fluid communication between said medical fluid container and said medical accessory by attachment of said medical accessory to said second open end and said valve member is motivated to close fluid communication between said medical fluid container and said medical accessory when said medical accessory is disengaged from said second open end.

2. The apparatus according to claim 1, wherein the biasing means comprises a spring.

3. The apparatus according to claim 2, wherein said second open end further includes an upstanding circumferential wall.

4. The apparatus according to claim 3 wherein the second open end further includes attachment means for releasably attaching said medical accessory to said second open end.

5. The apparatus according to claim 4 wherein said attachment means comprises at least one bayonet slot.

6. The apparatus according to claim 1, wherein said second open end further includes an upstanding circumferential wall.

7. The apparatus according to claim 6, wherein said second open end further includes an upstanding circumferential wall.

8. The apparatus according to claim 7 wherein the second open end further includes attachment means for releasably attaching said medical accessory to said second open end.

9. The apparatus according to claim 1 wherein said valve member further includes a valve nose extending axially outwardly from said valve seat for engagement with said medical accessory to open fluid communication.

10. The apparatus according to claim 9 wherein said valve seat further includes an upstanding annular ring on the surface distal to said hollow interior for fluid tight sealing engagement with said medical accessory.

11. A medical fluid container assembly, comprising:
a housing for containing medical fluid;
said housing having a hollow interior and an outlet for exit of medical fluid from said hollow interior; and
a fluid flow control means disposed within said outlet with fluid tight fit and adapted for fit with a medical accessory for controlling the flow of medical fluid materials between said hollow interior and said medical accessory;
said fluid flow control means comprising a valve member, a valve seat, biasing means operating on the valve member to bias said valve member against said valve seat for fluid tight fit, and whereby said valve member is motivated to open fluid communication between said hollow interior and said medical accessory when said medical accessory is attached to said fluid flow control means and said valve member is motivated to close fluid communication between said hollow interior and said medical accessory when said medical accessory is disengaged from said fluid flow control means.

12. The assembly according to claim 11 wherein said biasing means comprises a spring.

13. The assembly according to claim 12 wherein said fluid flow control means further includes an upstanding circumferential wall.

14. The assembly according to claim 13 wherein said fluid flow control means further includes an attachment means for releasably attaching said medical accessory to said fluid flow control means.

15. The assembly according to claim 14 wherein said attachment means comprises at least one bayonet slot.

16. The assembly according to claim 11 wherein said fluid flow control means further includes an upstanding circumferential wall.

17. The assembly according to claim 16 wherein said fluid flow control means further includes an attachment means for releasably attaching said medical accessory to said fluid flow control means.

18. An assembly according to claim 11 wherein said valve member further includes a valve nose extending axially outwardly from said valve seat for engagement with said medical accessory to open fluid communication.

19. An assembly according to claim 18 wherein said valve seat further includes an upstanding annular ring on the surface distal to said hollow interior for fluid tight sealing engagement with said medical accessory.

20. The apparatus according to claim 2 wherein said valve member further includes a valve nose extending axially outwardly from said valve seat for engagement with said medical accessory to open fluid communication.

21. The apparatus according to claim 3 wherein said valve member further includes a valve nose extending axially outwardly from said valve seat for engagement with said medical accessory to open fluid communication.

22. The apparatus according to claim 4 wherein said valve member further includes a valve nose extending axially outwardly from said valve seat for engagement with said medical accessory to open fluid communication.

23. The apparatus according to claim 5 wherein said valve member further includes a valve nose extending axially outwardly from said valve seat for engagement with said medical accessory to open fluid communication.

24. The apparatus according to claim 6 wherein said valve member further includes a valve nose extending axially outwardly from said valve seat for engagement with said medical accessory to open fluid communication.

25. The apparatus according to claim 7 wherein said valve member further includes a valve nose extending axially outwardly from said valve seat for engagement with said medical accessory to open fluid communication.

26. The apparatus according to claim 8 wherein said valve member further includes a valve nose extending axially outwardly from said valve seat for engagement with said medical accessory to open fluid communication.

27. An assembly according to claim 12 wherein said valve member further includes a valve nose extending axially outwardly from said valve seat for engagement with said medical accessory to open fluid communication.

28. An assembly according to claim 13 wherein said valve member further includes a valve nose extending axially outwardly from said valve seat for engagement with said medical accessory to open fluid communication.

29. An assembly according to claim 14 wherein said valve member further includes a valve nose extending axially outwardly from said valve seat for engagement with said medical accessory to open fluid communication.

30. An assembly according to claim 15 wherein said valve member further includes a valve nose extending axially outwardly from said valve seat for engagement with said medical accessory to open fluid communication.

31. An assembly according to claim 16 wherein said valve member further includes a valve nose extending axially outwardly from said valve seat for engagement with said medical accessory to open fluid communication.

32. An assembly according to claim 17 wherein said valve member further includes a valve nose extending axially outwardly from said valve seat for engagement with said medical accessory to open fluid communication.
